SEQUENCE LISTING (15 sequences)

SEQ ID NO: 1 - DNA, 336 bp, Phyllotreta cruciferae
aaggctgcgggtgaacttcaaaacgcccctggcggcggccagcgcccgcatcgagctccaccaatacaaaatcaacgagtcggtgatcacctgctacttcgcccagcccgtcacgcccgtcaagaaccccaacctgcagccgcccgccccatacaagcagttcctcatctcgccgcccgcctcgccgcccgtcgactgggagccgcggcccgagggcgagcccatcatcaaccacgacctgctggcggcgctggccaccctgagtcccgggggggcgcacgagttgcaccccccgtcgcccgggcagccgtctatagtggttcatacggcgttggg

SEQ ID NO: 2 - DNA, 347 bp, Phyllotreta cruciferae
cgtggacaccacgttcaccataagggtggtggagccgctgaagagcggcttcagcgagctgacccccaagcccagcaggggcaacacggggaagaaaggctatggcagcggcagggagactttgaggttcaaggcggatggtaacgcggaggtgcaggagcaggacgacgccatggaggcgggcgtggagaagatcaatgggatactggagtcctttatgggtataaatgattcggagctggcttcgcagatatgggatctttcggttgataagaagaactcgatggactttgcggaagcggtggacgactcggagttggctgtgtttgagttcacggatgagctga

SEQ ID NO: 3 - DNA, 353 bp, Phyllotreta cruciferae
aaatggcagccaccagacgattgcaaaaagaactaggagacattagaaattctggattaaaatcattccgtgatattcaagtggacgaaactaatattctaacttggcaaggtcttatagttccggataatccgccttacaacaaaggagcttttaaaatagaaattaatttccccgcagaatatccttttaagccgccaaaaataaattttaagacgaaaatttatcatcctaatatagatgaaaagggccaggtatgtctgcccattataagtgccgaaaattggaaacccgccactaaaacggaacaagtgatccaagcattggttgctcttgtgaacgagccagaaccg

SEQ ID NO: 4 - DNA, 300 bp, Phyllotreta cruciferae
tcaccaagagcttcggaggggctgggggttacctagccggatccaaggagtttataacgtttataagggagcatagtcacgcttcgcgacacgcttgggcgatgtccccgccagtggcggcccagatcatatcggtgattaaaattatcctgggtaaagacggcaccaatgaaggccagaggaggattgaaaacttggcgaggaacactcgatatttcaggctgagattggagcaaatggggttgatagtgcatggaaacgaagactcccctgttgttcccattttggtgtatctctact

SEQ ID NO: 5 - DNA, 299 bp, Phyllotreta striolata
tcaccaagagcttcggaggggctgggggttacctagccggatccaaggagtttataacgtttataagggagcatagtcacgcttcgcgacacgcttgggcgatgtccccgccagtggcggcccagatcatatcggtgattaaaattatcctgggtaaagacggcaccaatgaaggccagaggaggattgaaaacttggcgaggaacactcgatatttcaggctgagattggagcaaatggggttgatagtgcatggaaacgaagactcccctgttgttcctattttggtgtatctctac

SEQ ID NO: 6 - DNA, 300 bp, Phyllotreta cruciferae
agatcatatgcgcaacatcgttccgattcttttagataccaaaataacgaacgaatacgataagatgcgcatcatagctttgtacgctatgaccaaaaatggaataaccgaggaaaatcttacaaagctagcaacgcacgcccaaattaaagataaacaaactattgctaaccttcagctcttgggagttaatgtcattaatgatggtggccctagaaaaaagatctacaccgtaccgcgcaaagagcgaattacagaacaaacttatcagatgtcgagatggacacctgttattaaaga

SEQ ID NO: 7 - DNA, 329 bp, Phyllotreta cruciferae
tgtacatacctgccgttagtggttatttaaatttgtatttgagcctgcccgaagttcagagacttctcggtttacatgctgaacttttttacattcgaaaaggagttatcaacaattacgctctcaattttgttgtaccaataccatccaacatcgattcactatgtttcacgtgggaaagcctaacacctggacaaccagtacattattcaattcaagtggattcctcgaatgcagccgccttgccaaccccaaaattgaacatatcagacgccggagtgatccctaacaccgtacaaaccttccaaatatcattaccctgcaccggg

SEQ ID NO: 8 - DNA, 300 bp, Phyllotreta cruciferae
attacgcggtgaaggagttatcgacgaaagttctgctggtgttcatcacggcgctattcaccgttcgggggctgcagaagctgagggaggctctgaaattgcccccggggccatgggggctgcccatactgggatcgctgcccttcctcaaaggcgatttgcacctgcactacagggacctgacgcagaagtacgggtcgttgatttccaccagattgggctcgcaattgatcgttgttttaagtgattacaagatgatcagggatacgtttcgtaaggaggatttcaccggtagacctt

SEQ ID NO: 9 - DNA, 301 bp, Phyllotreta striolata
aattacgctgtgaaggagttgtcgacgaaagttctgctggtgttcatcacagcgctattcgccgcccgggggctgcagaagctgagggaggccctgaaattgccaccaggaccgtgggggctgcccatactgggatcgctgccctttctcaaaggcgatctgcacctgcactacagggacctgacgcagaagtacggctcgttgatatccaccagattgggttcgcaattgatcgttgttttaagtgattacaagatgatcagggatacgtttcgtaaggaggatttcaccggtagacctt

SEQ ID NO: 10 - DNA, 301 bp, Phyllotreta cruciferae
cgcttcgcaaaacgtccaatgaggtcaataaagtaactaacaggaacagttcctctcgctacaacgtaatttaaagccaccctgtacacgccgacagactggaggggcacaaggtccctttgctatccacggcggatccccacacgtcgcggctgacgccgggctccagtccggacatggacgatgggggcaagcggcaggccaccagggtgttcaagaagagctcccccaacgggaagatcaccgtctatttggggaagagggacttcgtcgatcacatatcacacgtggatcccataga

SEQ ID NO: 11 - DNA, 366 bp, Phyllotreta cruciferae
attcgcggacatgcgtacagtatcacattagttaaatatgttgatatagcaacgccaaatcaagttggaaaaattccattattacgattgagaaatccatggggtaatgaagccgaatggaatggaccatggagtgatcaatcacccgaatggcgatttattcctgatcatgaaaaagaagagcttggtttgacatttgataaagatggtgaattttggatgtcttttcatgattttcgaaaatattttacgaatttagagatatgtaatttaaatccagattcattaactgaagatgatttaaattcgggtaaaaaaaaatgggaaatgagtgtatttgagggtgaatgggtgcgtggtgttact

SEQ ID NO: 12 - DNA, 300 bp, Phyllotreta cruciferae
cgaatacatcaaagacatgagcgagatcgtcataaaggacatgaaaaactacgggctgtgcgttttagataacttcctgggggctgaaaggggcaggaacgtcttgtcggaggtgttgaccatggagagtgaaggagttttcagggacgggcaattggtgtcgtccaaaggggacgaagacagtaagactattcgaagcgatcaaatctgctgggtacacggtaaggagcccaactgtcccaacatcgggtatctggttagtaaggtcgattcggtcatcaccagggccaatcgaaggga

SEQ ID NO: 13 - DNA, 300 bp, Phyllotreta striolata
cgagtacatcaaagacatgagcgagatcgtcataaaggacatgagaaactacggtttgtgcgtcttagataacttcctgggcagcgaacggggcaggaacgtcctctccgaggtgttgctaatggagcgagaaggggtcttcagagacgggcaattggtgtcctccaaaggggacgaagacagcaagactatacgaagcgaccagatttgctgggtgcacggtaaagagcccaattgccccaacatcgggtatctcgtgagtaaggtcgatgctgttattacgagggccaatcggaggga

SEQ ID NO: 14 - DNA, 280 bp, Phyllotreta cruciferae
ctagaatttgcaattatggcgagttttcgttatttttcaaccgctcttgtactcttggcgatagtttggaaggcatcagccctgataggctgccaaataaaccccttcaaggaaaacgcgcctctgctggtataccccgaaaacaccacgataatttaccctgagcatagcagcaagtacatctctgtgaacccaggacaatcggtgaagtttgcttgtcccgaaagccgagtaaacttaggcgatagttcgattcccaaccaagtaactgccgtttgcg

SEQ ID NO: 15 - DNA, 280 bp, Phyllotreta striolata
gcaaaccgcattatacagctactacgacctgacgcgagccataggctcccgcggcaagtcaccccgtcgcccccaattcgccgaagaccgggacttctacaagctaaacggccgaaccgtcaaccaattgtacctgaggaagacgcagaggaagaccgtcaacgaattgctcggcttggacgactacgatacgaggtacatcaaaaacggggagctgtacttcctggcaaggggccacctgacggcgtacgcggatttcatctacccggcgctgcagaag

(19) United States
(12) Patent Application Publication
(10) Pub. No.: US 2022/0225622 A1
(43) Pub. Date: Jul. 21, 2022

(54) Flea Beetle-Specific RNAI-Based Pesticides

(71) Applicant: University of Manitoba, Winnipeg (CA)

(72) Inventor: Steve Whyard, Winnipeg (CA)

(21) Appl. No.: 17/605,788

(22) PCT Filed: Apr. 14, 2020
(86) PCT No.: PCT/CA2020/050497

§ 371 Date: Oct. 22, 2021

Related U.S. Application Data
(60) Provisional application No. 62/837,958, filed on Apr. 24, 2019.

(51) Int. Cl. A01N 63/60

(52) U.S. Cl. CPC A01N 63/60

(57) ABSTRACT

Flea beetles (species of the genus Phyllotreta) are serious pests of cruciferous crops, causing millions of dollars losses each year, despite the use of chemical pesticides. Our current pesticides are broad-spectrum in their activity, killing not just the pests, but also many non-target species, including beneficial insects such as pollinators and predators. Here, an alternative set of pesticides, based on double-stranded RNA (dsRNA), is shown to be effective at killing flea beetles when the dsRNAs are applied to a leaf surface and fed to the insects. Insects that fed on leaves sprayed with the dsRNAs died within 8 days of first exposure, but also showed reduced feeding activity within several days, thereby reducing feeding damage to the plants. Importantly, the dsRNAs were designed to be specific for the flea beetles, and when delivered to non-target beetles, no adverse effects were observed, illustrating the specificity of this new type of pesticide.

### PRIOR APPLICATION INFORMATION

The instant application claims the benefit of U.S. Provisional Patent Application 62/837,958, filed Apr. 24, 2019 and entitled “FLEA BEETLE-SPECIFIC RNAi-BASED PESTICIDES”, the entire contents of which are incorporated herein by reference.

### BACKGROUND OF THE INVENTION

Canola, cabbage, broccoli, and other cruciferous crops are attacked by a variety of insect pests, but flea beetles (Coleoptera: Chrysomelidae: Galerucinae: Alticini), are the most damaging, causing millions of dollars damage annually in those countries that grow these crops extensively. Neonicotinoid insecticide seed coatings, and pyrethroid, carbamate, and organophosphate foliar insecticides are used to control flea beetles, but these broad-spectrum insecticides not only kill the pest insects, but also kill non-target beneficial insects and can also adversely affect vertebrate species. With the continued use of these insecticides, there have been growing public concerns about the long-term adverse effects on non-target species and the increased risk of insecticide resistance developing in the pest populations, the latter of which results in increased frequencies of pesticide applications and ultimately reduced efficacy and increased damage.

To reduce our reliance on these broad-spectrum chemical insecticides and their associated problems, reduced-risk insecticides are needed. A technology that offers the promise of a reduced risk approach to insect pest control is RNA interference (RNAi). RNAi is a method of reducing or silencing a single gene's expression by applying double-stranded RNA (dsRNA) to the cells of most eukaryotic organisms. Once within the cell, the dsRNA is cleaved by an endonuclease called Dicer, which chops the dsRNA into short (typically 21-23 nt) interfering RNAs (siRNAs). These siRNAs are then bound to the RNA-induced silencing complex (RISC), which then scans all cellular messenger RNAs (mRNAs) for sequence matches to the siRNAs. If a match is found, an enzyme within RISC will cut the mRNA, thereby mediating the destruction of any RNA with identical sequence to the siRNAs, and silencing the gene's expression [1]. To mediate RNAi in vertebrates, siRNAs are used, as dsRNAs longer than 30 nt can induce interferon-based immune responses [2]. To mediate RNAi in invertebrates such as insects, longer dsRNAs are preferred, as they can generate a mixture of siRNAs that can bind to more of the target mRNA, and they are readily taken into the insects' cells. Some investigations have observed that dsRNAs less than 60 nt failed to enter the gut cells efficiently [3], but in some instances, 21 nt siRNAs were also effective at inducing systemic (body-wide) knockdown of the targeted mRNA [4].

Using bioinformatics tools, we can design dsRNAs to selectively target just one gene in an organism, and can also design dsRNA to target only one species or alternatively, a group of related species. With its incredible ability to reduce a gene's expression, RNAi is being considered for a great many applications, including a variety of crop protection technologies [5]. Transgenic plants that express species-specific insecticidal dsRNAs that target genes essential to the insects' metabolism, growth or development have the potential to kill insects that feed on the plants [6].

Because of RNAi's sequence-specificity, it can potentially be adapted to selectively target many different pest insects, without adversely affecting other organisms [7,8].

### SUMMARY OF THE INVENTION

According to a first aspect of the invention, there is provided a method of reducing feeding damage to a plant from a flea beetle of genus Phyllotreta comprising:

applying to a plant to be protected from flea beetles of the genus Phyllotreta an effective amount of a dsRNA to at least a portion of the plant to be protected, the nucleotide sequence of said dsRNA selected from the group consisting of:

(i) at least 21 consecutive nucleotides of the nucleotide sequence as set forth in SEQ ID No:1 (Pc1);

(ii) at least 21 consecutive nucleotides of the nucleotide sequence as set forth in SEQ ID No:2 (Pc2);

(iii) at least 21 consecutive nucleotides of the nucleotide sequence as set forth in SEQ ID No:3 (Pc3);

(iv) at least 21 consecutive nucleotides of the nucleotide sequence as set forth in SEQ ID No:4 (Pc4);

(v) at least 21 consecutive nucleotides of the nucleotide sequence as set forth in SEQ ID No:5 (Ps4);

(vi) at least 21 consecutive nucleotides of the nucleotide sequence as set forth in SEQ ID No:6 (Pc5);

(vii) at least 21 consecutive nucleotides of the nucleotide sequence as set forth in SEQ ID No:7 (Pc6);

(viii) at least 21 consecutive nucleotides of the nucleotide sequence as set forth in SEQ ID No:8 (Pc7);

(ix) at least 21 consecutive nucleotides of the nucleotide sequence as set forth in SEQ ID No:9 (Ps7);

(x) at least 21 consecutive nucleotides of the nucleotide sequence as set forth in SEQ ID No:10 (Pc8);

(xi) at least 21 consecutive nucleotides of the nucleotide sequence as set forth in SEQ ID No:11 (Pc9);

(xii) at least 21 consecutive nucleotides of the nucleotide sequence as set forth in SEQ ID No:12 (Pc10);

(xiii) at least 21 consecutive nucleotides of the nucleotide sequence as set forth in SEQ ID No:13 (Ps10);

and combinations thereof;

said dsRNA reducing feeding damage to the plant to be protected by reducing feeding activity of the flea beetles of the genus Phyllotreta following ingestion of said dsRNA by the flea beetles.

According to another aspect of the invention, there is provided a method of protecting a plant from feeding damage by a flea beetle of genus Phyllotreta comprising

applying to a plant to be protected from flea beetles of the genus Phyllotreta an effective amount of a dsRNA to at least a portion of the plant to be protected, the nucleotide sequence of said dsRNA selected from the group consisting of:

(i) at least 21 consecutive nucleotides of the nucleotide sequence as set forth in SEQ ID No:1 (Pc1);

(ii) at least 21 consecutive nucleotides of the nucleotide sequence as set forth in SEQ ID No:2 (Pc2);

(iii) at least 21 consecutive nucleotides of the nucleotide sequence as set forth in SEQ ID No:3 (Pc3);

(iv) at least 21 consecutive nucleotides of the nucleotide sequence as set forth in SEQ ID No:4 (Pc4);

(v) at least 21 consecutive nucleotides of the nucleotide sequence as set forth in SEQ ID No:5 (Ps4);

(vi) at least 21 consecutive nucleotides of the nucleotide sequence as set forth in SEQ ID No:6 (Pc5);

(vii) at least 21 consecutive nucleotides of the nucleotide sequence as set forth in SEQ ID No:7 (Pc6);

(viii) at least 21 consecutive nucleotides of the nucleotide sequence as set forth in SEQ ID No:8 (Pc7);

(ix) at least 21 consecutive nucleotides of the nucleotide sequence as set forth in SEQ ID No:9 (Ps7);

(x) at least 21 consecutive nucleotides of the nucleotide sequence as set forth in SEQ ID No:10 (Pc8);

(xi) at least 21 consecutive nucleotides of the nucleotide sequence as set forth in SEQ ID No:11 (Pc9);

(xii) at least 21 consecutive nucleotides of the nucleotide sequence as set forth in SEQ ID No:12 (Pc10);

(xiii) at least 21 consecutive nucleotides of the nucleotide sequence as set forth in SEQ ID No:13 (Ps10);

and combinations thereof;

said dsRNA protecting the plant to be protected by reducing feeding activity of the flea beetles of the genus Phyllotreta following ingestion of said dsRNA by the flea beetles.

According to a further aspect of the invention, there is provided a method of killing flea beetles of genus Phyllotreta comprising:

applying to a flea beetle of the genus Phyllotreta food source an effective amount of a dsRNA to at least a portion of the food source, the nucleotide sequence of said dsRNA selected from the group consisting of:

(i) at least 21 consecutive nucleotides of the nucleotide sequence as set forth in SEQ ID No:1 (Pc1);

(ii) at least 21 consecutive nucleotides of the nucleotide sequence as set forth in SEQ ID No:2 (Pc2);

(iii) at least 21 consecutive nucleotides of the nucleotide sequence as set forth in SEQ ID No:3 (Pc3);

(iv) at least 21 consecutive nucleotides of the nucleotide sequence as set forth in SEQ ID No:4 (Pc4);

(v) at least 21 consecutive nucleotides of the nucleotide sequence as set forth in SEQ ID No:5 (Ps4);

(vi) at least 21 consecutive nucleotides of the nucleotide sequence as set forth in SEQ ID No:6 (Pc5);

(vii) at least 21 consecutive nucleotides of the nucleotide sequence as set forth in SEQ ID No:7 (Pc6);

(viii) at least 21 consecutive nucleotides of the nucleotide sequence as set forth in SEQ ID No:8 (Pc7);

(ix) at least 21 consecutive nucleotides of the nucleotide sequence as set forth in SEQ ID No:9 (Ps7);

(x) at least 21 consecutive nucleotides of the nucleotide sequence as set forth in SEQ ID No:10 (Pc8);

(xi) at least 21 consecutive nucleotides of the nucleotide sequence as set forth in SEQ ID No:11 (Pc9);

(xii) at least 21 consecutive nucleotides of the nucleotide sequence as set forth in SEQ ID No:12 (Pc10);

(xiii) at least 21 consecutive nucleotides of the nucleotide sequence as set forth in SEQ ID No:13 (Ps10);

and combinations thereof;

said dsRNA killing the flea beetles of the genus Phyllotreta following ingestion of the food source comprising the dsRNA by the flea beetles of the genus Phyllotreta.

### BRIEF DESCRIPTION OF THE DRAWINGS

FIG. 1. Number of transcripts in P. cruciferae with moderate levels of expression (5<FPKM<20). A total of 654 of these sequences were expressed in larvae and both male and female adults. In P. striolata, similar numbers were observed, with 598 sequences expressed in both larvae and adults.

FIG. 2. Survival of P. cruciferae adults fed a single dose of 2.5 ng/mm² dsRNA over a 3-day period. The beetles were then fed untreated leaf discs and survival was monitored for 2 weeks. The values represent the percent survival of 20 treated insects, relative to the negative controls, which were fed gus-dsRNA. The dsRNAs shaded in brown are the 10 most potent, while the next 7 dsRNAs were effective at killing >85 of the beetles.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

In this study, the utility of RNAi technologies to kill flea beetles of the genus Phyllotreta was assessed, with the aim of developing topically-applied dsRNAs as an alternative to transgenic-mediated protection of the plants. First, it was demonstrated that dsRNA can be applied to canola leaves and when ingested by flea beetles, gene-specific knockdown of mRNA transcripts occurs. Secondly, a set of flea beetle of the genus Phyllotreta genes expressed in both larvae and adults was identified using transcriptomic analyses, and a subset of genes with no shared 21-nt lengths with other genes in NCBI's GenBank was subsequently identified. dsRNAs were prepared to >50 different flea beetle genes, and using feeding bioassays, a smaller subset of dsRNAs was identified that killed the flea beetles within 8 days. While this time-to-kill period is longer than that of conventional neurotoxin chemical pesticides, the species-specificity of the dsRNAs is a distinct advantage over the broad-spectrum activity of our currently used chemical pesticides. Furthermore, the feeding activity of the dsRNA-fed flea beetles was considerably reduced, relative to untreated controls, indicating that the crop plants will still be protected even before the insects die. A dose-dependency was observed for many of the dsRNAs, and some failed to kill the insects at even the highest doses tested, which indicates that not all dsRNAs are effective insecticides. dsRNAs were then mixed together, and some combinations provided improved efficacy, using less dsRNA in total to kill flea beetles.

As will be apparent to one of skill in the art, flea beetles of the genus Phyllotreta are known to be related by virtue of at least comparisons of cytochrome oxidase 1 (cox1) sequences. As discussed herein, P. cruciferae and P. striolata are two members of this genus.

In one aspect of the invention, there is provided a method of reducing feeding damage to a plant from a flea beetle of genus Phyllotreta comprising:

applying to a plant to be protected from flea beetles of the genus Phyllotreta an effective amount of a dsRNA to at least a portion of the plant to be protected, the nucleotide sequence of said dsRNA selected from the group consisting of:

(i) at least 21 consecutive nucleotides of the nucleotide sequence as set forth in SEQ ID No:1 (Pc1);

(ii) at least 21 consecutive nucleotides of the nucleotide sequence as set forth in SEQ ID No:2 (Pc2);

(iii) at least 21 consecutive nucleotides of the nucleotide sequence as set forth in SEQ ID No:3 (Pc3);

(iv) at least 21 consecutive nucleotides of the nucleotide sequence as set forth in SEQ ID No:4 (Pc4);

(v) at least 21 consecutive nucleotides of the nucleotide sequence as set forth in SEQ ID No:5 (Ps4);

(vi) at least 21 consecutive nucleotides of the nucleotide sequence as set forth in SEQ ID No:6 (Pc5);

(vii) at least 21 consecutive nucleotides of the nucleotide sequence as set forth in SEQ ID No:7 (Pc6);

(viii) at least 21 consecutive nucleotides of the nucleotide sequence as set forth in SEQ ID No:8 (Pc7);

(ix) at least 21 consecutive nucleotides of the nucleotide sequence as set forth in SEQ ID No:9 (Ps7);

(x) at least 21 consecutive nucleotides of the nucleotide sequence as set forth in SEQ ID No:10 (Pc8);

(xi) at least 21 consecutive nucleotides of the nucleotide sequence as set forth in SEQ ID No:11 (Pc9);

(xii) at least 21 consecutive nucleotides of the nucleotide sequence as set forth in SEQ ID No:12 (Pc10);

(xiii) at least 21 consecutive nucleotides of the nucleotide sequence as set forth in SEQ ID No:13 (Ps10);

and combinations thereof;

said dsRNA reducing feeding damage to the plant to be protected by reducing feeding activity of the flea beetles of the genus Phyllotreta following ingestion of said dsRNA by the flea beetles of the genus Phyllotreta.

In some embodiments, the nucleotide sequence of the selected dsRNA(s) may be at least 22 consecutive nucleotides, at least 23 consecutive nucleotides, at least 24 consecutive nucleotides, at least 25 consecutive nucleotides, at least 30 consecutive nucleotides, at least 35 consecutive nucleotides, at least 40 consecutive nucleotides, at least 45 consecutive nucleotides, at least 50 consecutive nucleotides, at least 60 consecutive nucleotides, at least 70 consecutive nucleotides, at least 80 consecutive nucleotides, at least 100 consecutive nucleotides, at least 125 consecutive nucleotides, at least 150 consecutive nucleotides, at least 175 nucleotides, at least 200 consecutive nucleotides or at least 250 consecutive nucleotides of any of the nucleotide sequences set forth in SEQ ID Nos:1-13.

As will be appreciated by one of skill in the art, an “effective amount” as used herein refers to an amount that is sufficient to achieve the desired result, that is, to reduce feeding activity of a flea beetle or to reduce damage to a plant from a flea beetle compared to an untreated control plant of similar size, age and type or to kill flea beetles. As such, an “effective amount” may depend on several factors, such as the environmental conditions, weather conditions, and the number of flea beetles that the plant or food source may encounter or is expected to encounter, which can be estimated using any of a variety of means known in the art.

As discussed herein, the dsRNA may be applied to at least one leaf of the plant to be protected. The dsRNA may be applied to the plant to be protected at a concentration of at least about 0.1 ng per mm², or at least about 0.5 ng per mm², that is, per mm² of plant material being coated, for example, one or more leaves.

Preferably, the flea beetle of the genus Phyllotreta is Phyllotreta cruciferae or Phyllotreta striolata.

Preferably, the plant is a cruciferous plant, although the plant may be any plant that is known to be or considered to be a potential food source of flea beetles of the genus Phyllotreta, for example, Phyllotreta cruciferae or Phyllotreta striolata.

The dsRNA may be a mixture of two or more dsRNA, for example, a mixture of a first dsRNA comprising at least 21 consecutive nucleotides of Pc1 and a second dsRNA comprising at least 21 consecutive nucleotides of Pc2 or Pc3; or a mixture of a first dsRNA comprising at least 21 consecutive nucleotides of Pc3 and a second dsRNA comprising at least 21 consecutive nucleotides of Pc1, Pc2, Pc4 or Pc5; or a mixture of a first dsRNA comprising at least 21 consecutive nucleotides of Pc2 and a second dsRNA comprising at least 21 consecutive nucleotides of Pc1, Pc3, Pc4, Pc5, Pc7, Pc9 or Pc10.

In another aspect of the invention, there is provided a method of protecting a plant from feeding damage by a flea beetle of genus Phyllotreta comprising

applying to a plant to be protected from flea beetles of the genus Phyllotreta an effective amount of a dsRNA to at least a portion of the plant to be protected, the nucleotide sequence of said dsRNA selected from the group consisting of:

(i) at least 21 consecutive nucleotides of the nucleotide sequence as set forth in SEQ ID No:1 (Pc1);

(ii) at least 21 consecutive nucleotides of the nucleotide sequence as set forth in SEQ ID No:2 (Pc2);

(iii) at least 21 consecutive nucleotides of the nucleotide sequence as set forth in SEQ ID No:3 (Pc3);

(iv) at least 21 consecutive nucleotides of the nucleotide sequence as set forth in SEQ ID No:4 (Pc4);

(v) at least 21 consecutive nucleotides of the nucleotide sequence as set forth in SEQ ID No:5 (Ps4);

(vi) at least 21 consecutive nucleotides of the nucleotide sequence as set forth in SEQ ID No:6 (Pc5);

(vii) at least 21 consecutive nucleotides of the nucleotide sequence as set forth in SEQ ID No:7 (Pc6);

(viii) at least 21 consecutive nucleotides of the nucleotide sequence as set forth in SEQ ID No:8 (Pc7);

(ix) at least 21 consecutive nucleotides of the nucleotide sequence as set forth in SEQ ID No:9 (Ps7);

(x) at least 21 consecutive nucleotides of the nucleotide sequence as set forth in SEQ ID No:10 (Pc8);

(xi) at least 21 consecutive nucleotides of the nucleotide sequence as set forth in SEQ ID No:11 (Pc9);

(xii) at least 21 consecutive nucleotides of the nucleotide sequence as set forth in SEQ ID No:12 (Pc10);

(xiii) at least 21 consecutive nucleotides of the nucleotide sequence as set forth in SEQ ID No:13 (Ps10);

and combinations thereof;

said dsRNA protecting the plant to be protected by reducing feeding activity of the flea beetles of the genus Phyllotreta following ingestion of said dsRNA by the flea beetles of the genus Phyllotreta.

As will be appreciated by one of skill in the art, the treated plant is protected from feeding damage compared to an untreated control plant of similar type, size and age subjected to feeding by a similar number of flea beetles of the genus Phyllotreta, preferably but not necessarily of the same species.

According to another aspect of the invention, there is provided a method of killing flea beetles of species Phyllotreta comprising:

applying to a flea beetle of the species Phyllotreta food source an effective amount of a dsRNA to at least a portion of the food source, the nucleotide sequence of said dsRNA selected from the group consisting of:

(i) at least 21 consecutive nucleotides of the nucleotide sequence as set forth in SEQ ID No:1 (Pc1);

(ii) at least 21 consecutive nucleotides of the nucleotide sequence as set forth in SEQ ID No:2 (Pc2);

(iii) at least 21 consecutive nucleotides of the nucleotide sequence as set forth in SEQ ID No:3 (Pc3);

(iv) at least 21 consecutive nucleotides of the nucleotide sequence as set forth in SEQ ID No:4 (Pc4);

(v) at least 21 consecutive nucleotides of the nucleotide sequence as set forth in SEQ ID No:5 (Ps4);

(vi) at least 21 consecutive nucleotides of the nucleotide sequence as set forth in SEQ ID No:6 (Pc5);

(vii) at least 21 consecutive nucleotides of the nucleotide sequence as set forth in SEQ ID No:7 (Pc6);

(viii) at least 21 consecutive nucleotides of the nucleotide sequence as set forth in SEQ ID No:8 (Pc7);

(ix) at least 21 consecutive nucleotides of the nucleotide sequence as set forth in SEQ ID No:9 (Ps7);

(x) at least 21 consecutive nucleotides of the nucleotide sequence as set forth in SEQ ID No:10 (Pc8);

(xi) at least 21 consecutive nucleotides of the nucleotide sequence as set forth in SEQ ID No:11 (Pc9);

(xii) at least 21 consecutive nucleotides of the nucleotide sequence as set forth in SEQ ID No:12 (Pc10);

(xiii) at least 21 consecutive nucleotides of the nucleotide sequence as set forth in SEQ ID No:13 (Ps10);

and combinations thereof;

said dsRNA killing the flea beetles of the genus Phyllotreta following ingestion of the food source comprising the dsRNA by the flea beetles of the genus Phyllotreta.

As discussed herein, the dsRNA was applied onto the leaves in just water. However, it is of note that there are a variety of formulation additives known in the art that act for example as spreaders, stickers and penetrants when applied to plants, for example, to leaves of plants. As one of skill in the art will appreciate, such formulation additives can be tested and optimized by one of routine skill in the art and are within the scope of the invention.

In some embodiments of the invention, the effective amount of the dsRNA is co-administered with an effective amount of a nuclease inhibitor.

Examples of known nuclease inhibitors include but are by no means limited to strong protein denaturants, such as guanidinium isothiocyanate; anionic polymers such as polyvinylsulfonic acid and protein-based nuclease inhibitors, such as : GamS Nuclease Inhibitor; Superase protein-based RNase inhibitor; and Recombinant RNase Inhibitor.

As will be appreciated by one of skill in the art, the challenge with using these types of inhibitors are many, but the most important is that most of the chemical are toxic to many organisms. Furthermore, the problem with using protein-based nuclease inhibitors is that they may be degraded by proteases in the gut of the insects feeding on them, and hence, they will fail to work.

In some embodiments, the nuclease inhibitor is a dsRNA targeted to a nuclease of flea beetles of species Phyllotreta.

In some embodiments, the nuclease inhibitor is selected from the group consisting of: at least 21 consecutive nucleotides of the nucleotide sequence as set forth in SEQ ID No: 14 (P. cruciferae DsRNase); and at least 21 consecutive nucleotides of the nucleotide sequence as set forth in SEQ ID No:15 (P. striolata DsRNase).

As will be appreciated by one of skill in the art, the nuclease inhibitor dsRNA can be co-administered or co-applied with the dsRNA. This does not necessarily mean that the dsRNA and the nuclease inhibitor dsRNA need to be co-formulated, although in some embodiments, they may be co-formulated, but merely that the dsRNA and nuclease inhibitor dsRNA are applied to the same plant(s) within a suitable time period.

In some embodiments, the nucleotide sequence of the nuclease inhibitor dsRNA(s) may be at least 22 consecutive nucleotides, at least 23 consecutive nucleotides, at least 24 consecutive nucleotides, at least 25 consecutive nucleotides, at least 30 consecutive nucleotides, at least 35 consecutive nucleotides, at least 40 consecutive nucleotides, at least 45 consecutive nucleotides, at least 50 consecutive nucleotides, at least 60 consecutive nucleotides, at least 70 consecutive nucleotides, at least 80 consecutive nucleotides, at least 100 consecutive nucleotides, at least 125 consecutive nucleotides, at least 150 consecutive nucleotides, at least 175 nucleotides, at least 200 consecutive nucleotides or at least 250 consecutive nucleotides of any of the nucleotide sequences set forth in SEQ ID No:14 or SEQ ID No: 15.

As will be appreciated by one of skill in the art, given that dsRNA can be fully degraded by nucleases within 30 minutes inside the gut of the beetles, it was not anticipated that feeding the insects with a nuclease-specific dsRNA would have any measurable impact on nuclease levels in the gut. Furthermore, it was anticipated that multiple nucleases are secreted into the gut of the beetles, so the reduction of a single nuclease's activity was not expected to have a significant impact on the protection of the dsRNA in the gut.

Furthermore, four nucleases were identified in the beetle with similarity to a nuclease found in the potato beetle, and it was not obvious which nuclease would be the best one to target.

As discussed herein, it was surprising how well the nuclease dsRNA improved the speed of kill of the flea beetles with some of the dsRNAs. Faster kill is particularly important, given that RNAi is still relatively slow compared to chemical pesticides (days vs hours, respectively), and a faster kill rate with the nuclease dsRNA means that fewer insects are continuing to feed until they die, meaning less damage to the protected plants.

As discussed herein, adding the nuclease dsRNA improves the efficacy of all of the insecticidal dsRNAs. For example, inclusion of the nuclease dsRNA in the mix, allows for reduction in the dose or effective amount of the insecticidal dsRNA, thereby saving on resources/costs.

The invention will now be further elucidated and/or explained by way of examples; however, the invention is not necessarily limited to the examples.

### Example 1. Ingested dsRNA Reduces Gene-specific Target mRNAs in Phyllotreta cruciferae and P. striolata in a Species-specific Manner

Two genes that have been observed to be susceptible to RNAi-mediated knockdown in other insects are the genes encoding snf7 and vATPase [2,4]. Sequence alignments of several insect species' genes were used to design degenerate primers, which were then used to PCR amplify portions of the snf7 and vATPase genes from both P. cruciferae and P. striolata. The PCR products were sequenced, and the snf7 sequences in the two species were 90% identical, and shared 53 (out of a possible maximum of 390) different 21-nt lengths. The vATPase sequences in the two species were 91% identical, and 21 (out of a possible maximum 495) shared 21-nt lengths. Using in vitro transcription, double-stranded RNAs were prepared for all 4 gene fragments. The dsRNAs were applied to 5 mm diameter canola leaf discs, and fed to 3 groups of 5 flea beetles every day, for 3 days. The beetles were then sacrificed and RNA was extracted from individual insects. The RNA was reverse transcribed to produce cDNA, which was used to measure transcript levels using qRT-PCR, which were compared to actin transcripts as a reference gene. In all individuals, snf7 and vATPase transcripts were reduced using the species-specific dsRNA, and intermediate levels of transcript knockdown were observed when the insects were fed dsRNA from the heterologous species as shown in Table 1. Negative controls were insects fed with a non-insect-specific gus-dsRNA, and no knockdown of the two target genes was observed. Specifically, ingested dsRNAs by flea beetles results in knockdown of targeted transcripts in the insects after 3 days. Each Insect ingested approximately 0.5 ng of dsRNA (based on 2.5 ng applied to each leaf disc, which was fully consumed by groups of 5 insects). The values represent the means and standard errors for 15 individual beetles. As can be seen in Table 1, the dsRNAs were more effective against the species from which they were derived, but also showed cross-species reactivity.

### Example 2. Transcriptomic Analyses Identified Flea Beetle Genes that were Expressed in Both Larvae and Adults, and Lacked Shared 21-nt Lengths with Other Genes in Genbank

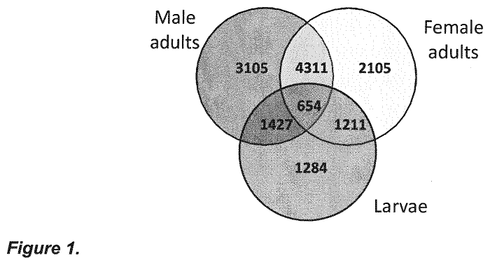

RNAseq was used to examine the transcriptomes of larvae, male adults, and female adults of P. cruciferae and P. striolata. In the absence of a flea beetle genome database, all transcripts were aligned to the annotated genome of another beetle, Tribolium castaneum. Sequences with moderate levels of expression were selected for comparisons across all three transcriptomes (larva, adult male, adult female), to identify genes that were expressed in all three. A total of 654 and 598 transcripts were identified in P. cruciferae and P. striolata that conformed to those criteria (FIG. 1). Of these sequences, all were used to query GenBank to identify those sequences that lack shared 21-nt identities with other species. In P. cruciferae, a total of 143 sequences that shared no 21 nt overlaps with any sequences in GenBank were identified, and in P. striolata, a total of 96 sequences were identified. They shared 74 of these genes, with 6 of them having no shared 21 nt overlaps between them. The remaining 68 genes shared between 2 and 52 21 nt overlaps, and could potentially be used to control both species. Given that these two species are within the same genus and closely related to each other phylogenetically, it is not surprising that of all the genes we identified as Phyllotreta-specific, they shared at least one or more 21 nts between these two species. Furthermore, as will be appreciated by one of skill in the art, the two species are both serious pests, developing a dsRNA that controlled only one may not be particularly useful.

### Example 3. Topically-applied dsRNA to Canola Leaves Kill Feeding Flea Beetles

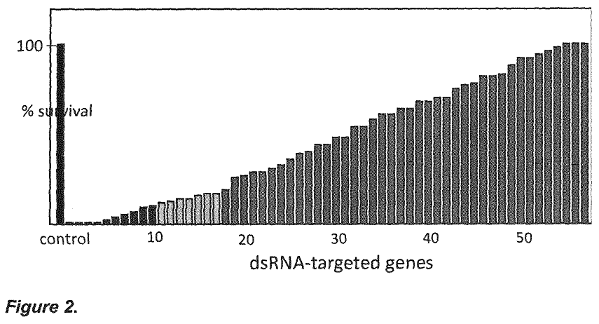

A subset of 56 of the flea beetle-specific dsRNAs were used to prepare gene-specific dsRNAs. The dsRNAs were applied topically to canola leaf discs (5 mm diameter) and fed to groups of 5 flea beetles. Each dsRNA treatment was replicated 4 times (for a total of 20 beetles) screened at 2.5 ng/mm². Of the first 56 dsRNAs screened, 25 (44%) failed to kill more than 50% of the beetles, and 9 (16%) failed to kill >10% of the beetles over a 2-week period, which indicated that not all dsRNAs are effective insecticides for these species (FIG. 2). Some of these ineffective dsRNAs may have failed to knock down the target gene's transcripts for various reasons. For example, if the gene is highly expressed, the dose of dsRNA may not have been sufficient to reduce the transcript levels sufficiently. While the genes selected for targeting were considered to be moderately expressed based on our transcriptomic analyses (with FPKM values between 5 and 20), some of the genes' expression levels may have exceeded that FPKM range during the feeding bioassays, perhaps due to either their normal cycling patterns or their ability to be closely regulate their expression, thereby countering the dsRNA's impact. The half-life of the encoded protein of the targeted gene can also affect RNAi efficacy; genes encoding proteins with long half-lives may not show any impact of RNAi unless dsRNA is delivered over a prolonged period. The values represent the percent survival of 20 treated insects, relative to the negative controls, which were fed gus-dsRNA. The dsRNAs shaded in brown are the 10 most potent, while the next 7 dsRNAs were effective at killing >85 of the beetles.

As discussed above, ten of the dsRNAs were highly effective at killing beetles, with >80% of the beetles dead within 7 days of treatment of 2.5 ng/mm². By day 14, all beetles in these 10 dsRNA treatments were dead. A dose dependency was observed for these 10 dsRNAs. Specifically, Table 2A shows mortality of P. cruciferae beetles after feeding on the ten most effective dsRNAs, using two different doses. Beetles were fed only a single ds-RNA treated leaf disc, and then fed untreated leaf discs thereafter. The values represent the means and standard errors for 4 treatments of 10 beetles each. Gus-dsRNA was used a negative control (line 1) and all mortalities below are relative to the negative control. Table 2 shows mortality of P. striolata beetles after feeding on the ten most effective dsRNAs, using two different doses. The values represent the means and standard errors for 4 treatments of 10 beetles each. Gus-dsRNA was used a negative control (line 1) and all mortalities below are relative to the negative control. These 10 genes are the likely orthologues of those listed in Table 2A, as the percent identities were all greater than 80% and all 10 shared >4 21 mers with P. cruciferae orthologues. These 10 dsRNAs still killed >80% of the beetles when 0.5 ng dsRNA/mm² was applied to the leaves.

The cross-reactivity of the 10 dsRNAs were tested on the other species, to determine whether the dsRNAs could be used to control both pest species. The dsRNA sequence identities between the two species ranged from 87% to 100%, sharing between 20 and 280 21 mers, across the ˜300 nt lengths of the dsRNAs. As shown in Table 3, insecticidal activity and the extent of transcript knockdown of each dsRNA on both species of flea beetles. Beetles were fed leaf discs coated with 1 ng/mm². The degree of cross-reactivity in insecticidal activity shows a good correlation with the number of shared 21 mers; sequences with 100% identity between the two species were equally effective at killing both species, whereas dsRNAs with reduced sequence identity, and therefore fewer shared 21 mers, were less effective at killing the other flea beetle species. Nevertheless, these dsRNAs with fewer shared 21 mers were still able to kill more than 50% of the beetles, indicating that could still be used to control both species. Alternatively, dsRNAs specific to each of the two different flea beetles could be combined to provided effective control of both pest species.

### Example 4. Topically-applied dsRNAs on Canola Leaves Reduces Leaf Consumption by Flea Beetles, Before Death of Beetles is Observed

While dsRNAs fed to flea beetles took >5 days to kill flea beetles, feeding on leaves was diminished by day 3, and leaf consumption was reduced by more than 50% over a 6-day period for the 10 most effective dsRNAs, as shown in Table 4. Five flea beetles per replicate were fed 5 mm diameter leaf discs with 2.5 ng/mm² dsRNA every third day for 9 days, and then fed untreated leaf discs thereafter. Gus-dsRNA was used as the negative control, and represents 100% leaf consumption. Untreated leaf discs showed similar levels of consumption, relative to the negative control gus-dsRNA. Uneaten portions of leaf discs were photographed and Image-J digital analysis was used to calculate the amount of leaf consumed. Values represent the average of 4 replicates of five beetles. The amount of leaf consumed is expressed in square millimeters and the percent eaten (in parentheses) is relative to the gus-dsRNA negative control treatments. No statistical difference was observed between gus-dsRNA and untreated leaf discs (t-test, p>0.5).

These results indicate that while dsRNAs are not as fast-acting as conventional neurotoxic pesticides, they can nevertheless protect plants from feeding damage by these herbivorous insects. Even though the dsRNAs did not immediately kill the insects, their feeding rates were sufficiently reduced as the dsRNAs took effect, thereby protecting the plants from extensive damage.

### Example 5. Combinations of dsRNAs Result in Synergism of Insecticidal Activity

DsRNAs were mixed in pairwise combinations and synergism was observed for a selection of these combinations, where a greater number of insects were killed using lower doses of the dsRNA than deaths using single dsRNAs at higher doses as shown in Table 5. Pairwise combinations can permit the use of less dsRNA applications, and can also permit multiple genes' transcripts to be targeted, to reduce possible resistance mechanisms from developing as quickly. The values represent the mortality of 8 groups of 5 beetles, relative to the negative control treatments (gus-dsRNA). Pairings with strong (++++), moderate (+++), or minor (++) synergism are indicated. The following combinations, showing effective synergy, enhancing the efficacy of the dsRNAs, killing flea beetles at considerably lower doses were:

Pc1 with Pc2 and Pc3;
Pc2 with Pc1, Pc3, Pc4, Pc5, Pc7, Pc9, and Pc10
Pc3 with Pc1, Pc2, Pc4, and Pc5
Interestingly, synergisms were observed for some pairings of dsRNAs and not others. It is worth noting that all of the most potent pairings involved genes associated with two different cellular mechanisms or pathways. In such instances, the affected cells would have endured perturbations to two different processes, which would be more effective at impairing cell function faster than simply targeting two genes involved in regulating the same cellular process/pathway.

### Example 6. The flea beetle dsRNA showed No Negative Impacts On Five Other Insects Typically Found within Canola Crops that are Predators of Flea Beetles

Insects were collected from canola fields in Manitoba and were identified using taxonomic keys. Two beetle species were selected that were known predators of flea beetles, and they were injected with 10× the dose required to kill flea beetles (i.e. 1 ng/mg fresh weight). Injection of two ladybird beetles with Pc1, Pc2, Pc3, or Pc4 dsRNA (10 ng/mg fresh weight) did not impact survivorship or reproductive rates. Between 25 and 30 insects were injected for each treatment, but those that died within 12 h of the injection were discarded, as the death was attributed to the injection injury, and not to dsRNA. No significant difference in mortality rates are seen between the negative control gus-dsRNA (a bacterial gene) and the insect dsRNAs (P>0.5, t test), except for the Coccinella septempunctata that were injected with their own species-specific dsRNA (P<0.05, t test). Survivors of the dsRNA treatments were provided two mates and the number of larvae that hatched from eggs was counted and standardized to the number of females per container.

Table 6 illustrates that no unrelated insects were negatively impacted by the flea beetle dsRNA itself, either in mortality assays, or in reproduction rates for groups of 10 beetles. For one ladybird beetle species, Coccinella septempunctata, a species-specific vATPase-dsRNA [9] was injected into the beetles, which resulted in 100% mortality of the insects in 5 days, indicating that dsRNA delivery can kill this insect if the dsRNA is designed to target the insect's specific genes.

Here, we have demonstrated that the dsRNAs can selectively kill the pest insects, but show no adverse effects on related non-target beetle species. Many of our currently used pesticides are broad-spectrum in their activity, killing both pests and beneficial/non-target species. The dsRNAs described here were selected on the basis of the lack of shared 21-mer matches to genes in Genbank, and their selectively for flea beetles was further confirmed by lack of negative impact on other beetles within the cropping system. In one study that examined the specificity of insecticidal activity of dsRNAs against the corm rootworm beetle, biological impacts were only observed in species that shared 21 mers with the pest species. In all cases, those non-target-species were phylogenetically closely related to the target pest species; specifically, another species within the same genus and a species within the same subfamily were the only species affected by the dsRNA. In these examples, we have observed flea-beetle specific insecticidal activity of the selected dsRNAs, with no observed negative impacts on other beetle species. Unlike broad-spectrum pesticides, which kill many off-target species, dsRNA-based pesticides show significant species-specificity, for the development of environmentally-friendlier pest control techniques.

### Example 7. Identification of Flea Beetle Nucleases from the Transcriptome Data

Gut nucleases can potentially degrade dsRNAs within the insect guts before the dsRNA can be taken up by cells to induce RNAi. Previous studies have demonstrated that co-delivery of nuclease-specific dsRNAs along with a second dsRNA will enhance the efficacy of the second dsRNA, after a few days' exposure to both dsRNAs.
A search of flea beetle transcriptomic data identified four genes in each species with partial sequence identity to nucleases in other insects. One gene in P. cruciferae showed the most similarity (66% nt identity) to a portion of a non-specific nuclease in the Colorado potato beetle, Leptinotarsa decemlineata (GenBank accession: KX652406.1. Similarly, in P. striolata, one gene showed 68% identity to a portion of this same gene in L. decemlineata. A dsRNA was designed to target 280 bp of the respective gene in each flea beetle. Each flea beetle dsRNA shared no 21-mers with that of the other species, and less than 50% identity with any other sequence in GenBank. This low level of sequence identity suggested that these genes identified in flea beetles were not necessary orthologues of the potato beetle, but nevertheless encoded distantly related nucleases.

### Example 8. RNAi Validation Confirmed that the Identified Genes Encoded dsRNases

Adult flea beetles were fed their respective putative nuclease-dsRNAs on canola leaf discs at a dose of 2.5 ng/mm² for three days. Three groups of five beetles were sacrificed on the third day, RNA extracted, and the extent of knockdown of each gene was assessed using qRT-PCR. In each species, transcripts were reduced by 81 and 84% in P. cruciferae and P. striolata, respectively. Guts were dissected from nuclease-dsRNA-treated and gus-dsRNA-treated (negative controls) beetles after 4 days of feeding, and the guts were soaked in PBS buffer overnight to permit the nucleases to diffuse into the buffer. These gut extracts were then incubated with 200 ng gus-dsRNA for 30 minutes to assess whether any gut nucleases exhibited an ability to degrade dsRNA. The extract-treated dsRNAs were loaded onto agarose gels, stained with ethidium bromide, and dsRNA was visualized with UV light. Gut extracts from the negative control beetles had completely degraded the gus-dsRNA, as no dsRNA was visible in the gel, whereas gut extracts treated with putative nuclease-specific dsRNA had only degraded 18-25% of the dsRNA, in the extracted collected from P. cruciferae and P. striolata guts, respectively. These results confirmed that the sequences identified were dsRNase-encoding genes.

### Example 9. Mixing dsRNase-dsRNA with Other dsRNAs Improved RNAi Efficiency

In other insects, knockdown of gut nucleases, achieved by delivering dsRNA targeting the nuclease transcripts, has been shown to improve RNAi efficiency for other dsRNAs. Here, the two species of flea beetles were fed their respective dsRNase-specific dsRNA along with the each of the 10 previously described insecticidal dsRNAs, and after 7 days, increased mortality rates and a shorter time to kill for all 10 dsRNAs tested, at half the dose, with the dsRNase-specific dsRNA (Table 7). These findings demonstrate that by co-delivering the dsRNase-specific dsRNA along with another insecticidal dsRNA, improved insecticidal action of the second dsRNA can be achieved.

DsRNA Sequences
Pc1 (= Psi; 100% identical in P. cruciferae and P. striolata),
SEQ ID No: 1:
AAGGCTGCGGGTGAACTTCAAAACGCCCCTGGCGGCGGCCAGCGCCCGCATCG
AGCTCCACCAATACAAAATCAACGAGTCGGTGATCACCTGCTACTTCGCCCAGC
CCGTCACGCCCGTCAAGAACCCCAACCTGCAGCCGCCCGCCCCATACAAGCAG
TTCCTCATCTCGCCGCCCGCCTCGCCGCCCGTCGACTGGGAGCCGCGGCCCGA
GGGCGAGCCCATCATCAACCACGACCTGCTGGCGGCGCTGGCCACCCTGAGTC
CCGGGGGGGCGCACGAGTTGCACCCCCCGTCGCCCGGGCAGCCGTCTATAGT
GGTTCATACGGCGTTGGG
Pc2 (= Ps2; 100% identical in P. cruciferae and P. striolata),
SEQ ID No: 2:
CGTGGACACCACGTTCACCATAAGGGTGGTGGAGCCGCTGAAGAGCGGCTTCA
GCGAGCTGACCCCCAAGCCCAGCAGGGGCAACACGGGGAAGAAAGGCTATGG
CAGCGGCAGGGAGACTTTGAGGTTCAAGGCGGATGGTAACGCGGAGGTGCAGG
AGCAGGACGACGCCATGGAGGCGGGCGTGGAGAAGATCAATGGGATACTGGAG
TCCTTTATGGGTATAAATGATTCGGAGCTGGCTTCGCAGATATGGGATCTTTCGG
TTGATAAGAAGAACTCGATGGACTTTGCGGAAGCGGTGGACGACTCGGAGTTGG
CTGTGTTTGAGTTCACGGATGAGCTGA
Pc3 (= Ps3; 100% identical in P. cruciferae and P. striolata),
SEQ ID No: 3:
AAATGGCAGCCACCAGACGATTGCAAAAAGAACTAGGAGACATTAGAAATTCTG
GATTAAAATCATTCCGTGATATTCAAGTGGACGAAACTAATATTCTAACTTGGCAA
GGTCTTATAGTTCCGGATAATCCGCCTTACAACAAAGGAGCTTTTAAAATAGAAA
TTAATTTCCCCGCAGAATATCCTTTTAAGCCGCCAAAAATAAATTTTAAGACGAAA
ATTTATCATCCTAATATAGATGAAAAGGGCCAGGTATGTCTGCCCATTATAAGTG
CCGAAAATTGGAAACCCGCCACTAAAACGGAACAAGTGATCCAAGCATTGGTTG
CTCTTGTGAACGAGCCAGAACCG
Pc4, SEQ ID No: 4:
TCACCAAGAGCTTCGGAGGGGCTGGGGGTTACCTAGCCGGATCCAAGGAGTTT
ATAACGTTTATAAGGGAGCATAGTCACGCTTCGCGACACGCTTGGGCGATGTCC
CCGCCAGTGGCGGCCCAGATCATATCGGTGATTAAAATTATCCTGGGTAAAGAC
GGCACCAATGAAGGCCAGAGGAGGATTGAAAACTTGGCGAGGAACACTCGATAT
TTCAGGCTGAGATTGGAGCAAATGGGGTTGATAGTGCATGGAAACGAAGACTCC
CCTGTTGTTCCCATTTTGGTGTATCTCTACT
Ps4 (99.7% identical to Pc4), SEQ ID No: 5:
TCACCAAGAGCTTCGGAGGGGCTGGGGGTTACCTAGCCGGATCCAAGGAGTTT
ATAACGTTTATAAGGGAGCATAGTCACGCTTCGCGACACGCTTGGGCGATGTCC
CCGCCAGTGGCGGCCCAGATCATATCGGTGATTAAAATTATCCTGGGTAAAGAC
GGCACCAATGAAGGCCAGAGGAGGATTGAAAACTTGGCGAGGAACACTCGATAT
TTCAGGCTGAGATTGGAGCAAATGGGGTTGATAGTGCATGGAAACGAAGACTCC
CCTGTTGTTCCTATTTTGGTGTATCTCTAC
Pc5 (= Ps5; 100% identical in P. cruciferae and P. striolata),
SEQ ID No: 6:
AGATCATATGCGCAACATCGTTCCGATTCTTTTAGATACCAAAATAACGAACGAAT
ACGATAAGATGCGCATCATAGCTTTGTACGCTATGACCAAAAATGGAATAACCGA
GGAAAATCTTACAAAGCTAGCAACGCACGCCCAAATTAAAGATAAACAAACTATT
GCTAACCTTCAGCTCTTGGGAGTTAATGTCATTAATGATGGTGGCCCTAGAAAAA
AGATCTACACCGTACCGCGCAAAGAGCGAATTACAGAACAAACTTATCAGATGTC
GAGATGGACACCTGTTATTAAAGA
Pc6 (= Ps6; 100% identical in P. cruciferae and P. striolata),
SEQ ID No: 7:
TGTACATACCTGCCGTTAGTGGTTATTTAAATTTGTATTTGAGCCTGCCCGAAGTT
CAGAGACTTCTCGGTTTACATGCTGAACTTTTTTACATTCGAAAAGGAGTTATCAA
CAATTACGCTCTCAATTTTGTTGTACCAATACCATCCAACATCGATTCACTATGTT
TCACGTGGGAAAGCCTAACACCTGGACAACCAGTACATTATTCAATTCAAGTGGA
TTCCTCGAATGCAGCCGCCTTGCCAACCCCAAAATTGAACATATCAGACGCCGG
AGTGATCCCTAACACCGTACAAACCTTCCAAATATCATTACCCTGCACCGGG
Pc7, SEQ ID No: 8:
ATTACGCGGTGAAGGAGTTATCGACGAAAGTTCTGCTGGTGTTCATCACGGCGC
TATTCACCGTTCGGGGGCTGCAGAAGCTGAGGGAGGCTCTGAAATTGCCCCCG
GGGCCATGGGGGCTGCCCATACTGGGATCGCTGCCCTTCCTCAAAGGCGATTT
GCACCTGCACTACAGGGACCTGACGCAGAAGTACGGGTCGTTGATTTCCACCAG
ATTGGGCTCGCAATTGATCGTTGTTTTAAGTGATTACAAGATGATCAGGGATACG
TTTCGTAAGGAGGATTTCACCGGTAGACCTT
Ps7 (94% identical to Pc7), SEQ ID No: 9:
AATTACGCTGTGAAGGAGTTGTCGACGAAAGTTCTGCTGGTGTTCATCACAGCG
CTATTCGCCGCCCGGGGGCTGCAGAAGCTGAGGGAGGCCCTGAAATTGCCACC
AGGACCGTGGGGGCTGCCCATACTGGGATCGCTGCCCTTTCTCAAAGGCGATC
TGCACCTGCACTACAGGGACCTGACGCAGAAGTACGGCTCGTTGATATCCACCA
GATTGGGTTCGCAATTGATCGTTGTTTTAAGTGATTACAAGATGATCAGGGATAC
GTTTCGTAAGGAGGATTTCACCGGTAGACCTT
Pc8 (= Ps8; 100% identical in P. cruciferae and P. striolata),
SEQ ID No: 10:
CGCTTCGCAAAACGTCCAATGAGGTCAATAAAGTAACTAACAGGAACAGTTCCTC
TCGCTACAACGTAATTTAAAGCCACCCTGTACACGCCGACAGACTGGAGGGGCA
CAAGGTCCCTTTGCTATCCACGGCGGATCCCCACACGTCGCGGCTGACGCCGG
GCTCCAGTCCGGACATGGACGATGGGGGCAAGCGGCAGGCCACCAGGGTGTT
CAAGAAGAGCTCCCCCAACGGGAAGATCACCGTCTATTTGGGGAAGAGGGACTT
CGTCGATCACATATCACACGTGGATCCCATAGA
Pc9 (= Ps9; 100% identical in P. cruciferae and P. striolata),
SEQ ID No: 11:
ATTCGCGGACATGCGTACAGTATCACATTAGTTAAATATGTTGATATAGCAACGC
CAAATCAAGTTGGAAAAATTCCATTATTACGATTGAGAAATCCATGGGGTAATGA
AGCCGAATGGAATGGACCATGGAGTGATCAATCACCCGAATGGCGATTTATTCC
TGATCATGAAAAAGAAGAGCTTGGTTTGACATTTGATAAAGATGGTGAATTTTGG
ATGTCTTTTCATGATTTTCGAAAATATTTTACGAATTTAGAGATATGTAATTTAAAT
CCAGATTCATTAACTGAAGATGATTTAAATTCGGGTAAAAAAAAATGGGAAATGA
GTGTATTTGAGGGTGAATGGGTGCGTGGTGTTACT
Pc10, SEQ ID No: 12:
CGAATACATCAAAGACATGAGCGAGATCGTCATAAAGGACATGAAAAACTACGG
GCTGTGCGTTTTAGATAACTTCCTGGGGGCTGAAAGGGGCAGGAACGTCTTGTC
GGAGGTGTTGACCATGGAGAGTGAAGGAGTTTTCAGGGACGGGCAATTGGTGT
CGTCCAAAGGGGACGAAGACAGTAAGACTATTCGAAGCGATCAAATCTGCTGGG
TACACGGTAAGGAGCCCAACTGTCCCAACATCGGGTATCTGGTTAGTAAGGTCG
ATTCGGTCATCACCAGGGCCAATCGAAGGGA
Ps10 (87.4% identical to Pc10), SEQ ID No: 13:
CGAGTACATCAAAGACATGAGCGAGATCGTCATAAAGGACATGAGAAACTACGG
TTTGTGCGTCTTAGATAACTTCCTGGGCAGCGAACGGGGCAGGAACGTCCTCTC
CGAGGTGTTGCTAATGGAGCGAGAAGGGGTCTTCAGAGACGGGCAATTGGTGT
CCTCCAAAGGGGACGAAGACAGCAAGACTATACGAAGCGACCAGATTTGCTGG
GTGCACGGTAAAGAGCCCAATTGCCCCAACATCGGGTATCTCGTGAGTAAGGTC
GATGCTGTTATTACGAGGGCCAATCGGAGGGA
P. cruciferae DsRNase dsRNA sequence (280 nt) SEQ ID No: 14
CTAGAATTTGCAATTATGGCGAGTTTTCGTTATTTTTCAACCGCTCTTGTACTCTT
GGCGATAGTTTGGAAGGCATCAGCCCTGATAGGCTGCCAAATAAACCCCTTCAA
GGAAAACGCGCCTCTGCTGGTATACCCCGAAAACACCACGATAATTTACCCTGA
GCATAGCAGCAAGTACATCTCTGTGAACCCAGGACAATCGGTGAAGTTTGCTTG
TCCCGAAAGCCGAGTAAACTTAGGCGATAGTTCGATTCCCAACCAAGTAACTGC
CGTTTGCG
P. striolata DsRNase dsRNA sequence (280 nt) SEQ ID No: 15
GCAAACCGCATTATACAGCTACTACGACCTGACGCGAGCCATAGGCTCCCGCGG
CAAGTCACCCCGTCGCCCCCAATTCGCCGAAGACCGGGACTTCTACAAGCTAAA
CGGCCGAACCGTCAACCAATTGTACCTGAGGAAGACGCAGAGGAAGACCGTCA
ACGAATTGCTCGGCTTGGACGACTACGATACGAGGTACATCAAAAACGGGGAGC
TGTACTTCCTGGCAAGGGGCCACCTGACGGCGTACGCGGATTTCATCTACCCGG
CGCTGCAGAAG

While the preferred embodiments of the invention have been described above, it will be recognized and understood that various modifications may be made therein, and the appended claims are intended to cover all such modifications which may fall within the spirit and scope of the invention.

TABLE 1
Ingested dsRNAs by flea beetles results in knockdown of targeted
transcripts in the insects after 3 days. Each Insect ingested
approximately 0.5 ng of dsRNA (based on 2.5 ng applied to
each leaf disc, which was fully consumed by groups of 5 insects).
The knockdown of transcripts was determined by qRT-PCR, using
actin as a reference gene and using gus-dsRNA as a negative
control treatment. The values represent the means and standard
errors for 15 individual beetles.

| | % transcript knockdown relative to gfp-dsRNA | | | |
|---|---|---|---|---|
| | DsRNA used: | | | |
| Species assayed | Pc-snf7 | Ps-snf7 | Pc-vATPase | Ps-vATPase |
| P. cruciferae (Pc) | 87 ± 5 | 23 ± 4 | 83 ± 7 | 15 ± 3 |
| P. striolata (Ps) | 26 ± 5 | 92 ± 6 | 18 ± 3 | 85 ± 6 |

TABLE 2A
Mortality of P. cruciferae beetles after feeding on the ten most effective
dsRNAs, using two different doses. Beetles were fed only a single ds-RNA
treated leaf disc, and then fed untreated leaf discs thereafter. The
values represent the means and standard errors for 4 treatments of 10
beetles each. Gus-dsRNA was used a negative control (line 1) and all
mortalities below are relative to the negative control.

| % mortality (relative to the negative control) | |
|---|---|
| 0.5 ng/mm² | 2.5 ng/mm² |

| DsRNA | Day 7 | Day 14 | Day 7 | Day 14 |
|---|---|---|---|---|
| Gus-dsRNA | 3 ± 1 | 8 ± 2 | 4 ± 1 | 7 ± 3 |
| (neg) | | | | |
| PC1 | 43 ± 4 | 90 ± 5 | 98 ± 1 | 100 |
| PC2 | 40 ± 5 | 87 ± 5 | 98 ± 1 | 100 |
| PC3 | 40 ± 4 | 87 ± 4 | 93 ± 1 | 100 |
| PC4 | 36 ± 5 | 83 ± 6 | 93 ± 1 | 100 |
| PC5 | 35 ± 5 | 83 ± 8 | 91 ± 1 | 100 |
| PC6 | 33 ± 3 | 80 ± 6 | 90 ± 2 | 100 |
| PC7 | 33 ± 4 | 78 ± 5 | 86 ± 3 | 100 |
| PC8 | 30 ± 3 | 76 ± 4 | 84 ± 3 | 100 |
| PC9 | 28 ± 5 | 76 ± 8 | 82 ± 4 | 100 |
| PC10 | 22 ± 6 | 73 ± 6 | 81 ± 5 | 100 |

TABLE 2B
Mortality of P. striolata beetles after feeding on the ten
most effective dsRNAs, using two different doses. The values
represent the means and standard errors for 4 treatments of 10
beetles each. Gus-dsRNA was used a negative control (line 1)
and all mortalities below are relative to the negative control.
These 10 genes are the likely orthologues of those listed in Table
2A, as the percent identities were all greater than 80% and all
10 shared >4 21mers with P. cruciferae orthologues.

| % mortality (relative to the negative control) | |
|---|---|
| 0.5 ng/mm² | 2.5 ng/mm² |

| DsRNA | Day 7 | Day 14 | Day 7 | Day 14 |
|---|---|---|---|---|
| Gus-dsRNA | 5 ± 2 | 9 ± 2 | 5 ± 1 | 8 ± 3 |
| (neg) | | | | |
| PS1 | 39 ± 5 | 88 ± 6 | 95 ± 3 | 100 |
| PS2 | 35 ± 5 | 85 ± 7 | 95 ± 3 | 100 |
| PS3 | 34 ± 4 | 85 ± 4 | 92 ± 4 | 100 |
| PS4 | 33 ± 4 | 84 ± 9 | 90 ± 5 | 100 |
| PS5 | 29 ± 6 | 83 ± 3 | 90 ± 4 | 100 |
| PS6 | 28 ± 2 | 81 ± 8 | 87 ± 9 | 100 |
| PS7 | 26 ± 4 | 79 ± 7 | 83 ± 5 | 100 |
| PS8 | 24 ± 5 | 74 ± 5 | 82 ± 6 | 100 |
| PS9 | 24 ± 3 | 70 ± 6 | 80 ± 6 | 100 |
| PS10 | 20 ± 3 | 72 ± 7 | 80 ± 7 | 100 |

TABLE 3
Insecticidal activity and the extent of transcript knockdown
of each dsRNA on both species of flea beetles. Beetles were
fed leaf discs coated with 1 ng/mm².

| | P. cruciferae | | | P. striolata | |
|---|---|---|---|---|---|
| | % transcript | % | # shared | % transcript | % |
| DsRNA | KD | mortality | 21mers | KD | mortality |
| Gus | 0 | 7 | — | 0 | 8 |
| Pc1 | 92 ± 4 | 100 ± 0 | 280 | 94 ± 2 | 99 ± 1 |
| Pc2 | 94 ± 3 | 100 ± 0 | 280 | 92 ± 4 | 98 ± 1 |
| Pc3 | 89 ± 5 | 94 ± 2 | 280 | 91 ± 3 | 97 ± 2 |
| Pc4 | 90 ± 3 | 96 ± 2 | 260 | 89 ± 3 | 95 ± 3 |
| Pc5 | 85 ± 6 | 93 ± 3 | 280 | 87 ± 4 | 96 ± 3 |
| Pc6 | 89 ± 5 | 93 ± 4 | 280 | 84 ± 4 | 94 ± 4 |
| Pc7 | 84 ± 7 | 89 ± 4 | 104 | 56 ± 6 | 84 ± 7 |
| Pc8 | 89 ± 5 | 89 ± 5 | 280 | 86 ± 6 | 90 ± 4 |
| Pc9 | 83 ± 7 | 90 ± 2 | 280 | 81 ± 5 | 83 ± 6 |
| Pc10 | 79 ± 6 | 86 ± 6 | 20 | 45 ± 5 | 59 ± 8 |
| Ps1 | 92 ± 4 | 100 ± 0 | 280 | 94 ± 2 | 99 ± 1 |
| Ps2 | 94 ± 3 | 100 ± 0 | 280 | 92 ± 4 | 98 ± 1 |
| Ps3 | 89 ± 5 | 94 ± 2 | 280 | 91 ± 3 | 97 ± 2 |
| Ps4 | 89 ± 6 | 94 ± 4 | 260 | 86 ± 3 | 92 ± 2 |
| Ps5 | 85 ± 6 | 93 ± 3 | 280 | 87 ± 4 | 96 ± 3 |
| Ps6 | 89 ± 5 | 89 ± 5 | 280 | 84 ± 4 | 94 ± 4 |
| Ps7 | 60 ± 6 | 81 ± 7 | 104 | 89 ± 5 | 93 ± 3 |
| Ps8 | 89 ± 5 | 89 ± 4 | 280 | 86 ± 6 | 90 ± 4 |
| Ps9 | 83 ± 7 | 90 ± 2 | 280 | 81 ± 5 | 83 ± 6 |
| Ps10 | 44 ± 6 | 54 ± 8 | 20 | 89 ± 2 | 85 ± 5 |

TABLE 4
Flea beetles show reduced leaf consumption after feeding on dsRNAs.
Five flea beetles per replicate were fed 5 mm diameter leaf discs with
2.5 ng/mm² dsRNA every third day for 9 days, and then fed untreated
leaf discs thereafter. Gus-dsRNA was used as the negative control,
and represents 100% leaf consumption. Untreated leaf discs showed
similar levels of consumption, relative to the negative control
gus-dsRNA. Uneaten portions of leaf discs were photographed and
Image-J digital analysis was used to calculate the amount of
leaf consumed. Values represent the average of 4 replicates of
five beetles. The amount of leaf consumed is expressed in square
millimeters and the percent eaten (in parentheses) is relative
to the gus-dsRNA negative control treatments. No statistical
difference was observed between gus-dsRNA and untreated leaf
discs (t-test, p > 0.5)

| | | |
|---|---|---|
| | Leaf | Leaf |
| | consumption | consumption |
| DsRNA | day 6 (%) | day 14 (%) |

P. cruciferae

| | | |
|---|---|---|
| None | 110 ± 11 | 250 ± 18 |

| | | | | |
|---|---|---|---|---|
| Gus | 105 ± 12 | (100) | 242 ± 16 | (100) |
| Pc1 | 33 ± 5 | (31) | 42 ± 4 | (17) |
| Pc2 | 29 ± 6 | (27) | 38 ± 5 | (16) |
| Pc3 | 32 ± 4 | (30) | 40 ± 6 | (17) |
| Pc4 | 45 ± 7 | (43) | 54 ± 4 | (22) |
| Pc5 | 36 ± 6 | (34) | 67 ± 5 | (28) |
| Pc6 | 41 ± 8 | (39) | 62 ± 4 | (26) |
| Pc7 | 35 ± 5 | (33) | 70 ± 6 | (29) |
| Pc8 | 46 ± 5 | (44) | 76 ± 6 | (31) |
| Pc9 | 42 ± 3 | (40) | 82 ± 5 | (34) |
| Pc10 | 50 ± 5 | (47) | 95 ± 8 | (39) |
| P. striolata | | | | |
| Gus | 94 ± 8 | (100) | 231 ± 17 | (100) |

| | | | |
|---|---|---|---|
| Ps1 | 29 ± 4 | 41 ± 5 | (18) |
| Ps2 | 21 ± 5 | 40 ± 4 | (17) |
| Ps3 | 35 ± 6 | 49 ± 6 | (21) |
| Ps4 | 27 ± 5 | 52 ± 4 | (23) |
| Ps5 | 38 ± 3 | 67 ± 5 | (29) |
| Ps6 | 47 ± 5 | 89 ± 8 | (39) |
| Ps7 | 27 ± 4 | 65 ± 7 | (28) |
| Ps8 | 42 ± 3 | 85 ± 7 | (37) |
| Ps9 | 40 ± 4 | 98 ± 10 | (42) |
| Ps10 | 45 ± 4 | 108 ± 7 | (47) |

TABLE 5
Some combinations of dsRNAs cause increased insect mortality,
compared to single treatments of equivalent doses. Flea beetles
were fed leaf discs once with dsRNAs alone at two different
doses, or combined at the two half doses. The values represent
the mortality of 8 groups of 5 beetles, relative to the negative
control treatments (gus-dsRNA). Pairings with strong (++++),
moderate (+++), or minor (++) synergism are indicated.

| | | | Synergism |
|---|---|---|---|
| | Dose ng/mm² | % mortality | pairing |
| Pc1 | 0.5 | 44 ± 5 | |
| Pc1 | 0.25 | 31 ± 4 | |
| Pc2 | 0.5 | 39 ± 4 | |
| Pc2 | 0.25 | 28 ± 3 | |
| Pc3 | 0.5 | 37 ± 5 | |
| Pc3 | 0.25 | 24 ± 3 | |
| Pc4 | 0.5 | 38 ± 5 | |
| Pc4 | 0.25 | 26 ± 4 | |
| Pc5 | 0.5 | 36 ± 3 | |
| Pc5 | 0.25 | 21 ± 3 | |
| Pc6 | 0.5 | 33 ± 5 | |
| Pc6 | 0.25 | 19 ± 4 | |
| Pc7 | 0.5 | 32 ± 3 | |
| Pc7 | 0.25 | 18 ± 2 | |
| Pc8 | 0.5 | 31 ± 3 | |
| Pc8 | 0.25 | 20 ± 3 | |
| Pc9 | 0.5 | 28 ± 4 | |
| Pc9 | 0.25 | 17 ± 3 | |
| Pc10 | 0.5 | 23 ± 3 | |
| Pc10 | 0.25 | 15 ± 2 | |
| Pc1 + Pc2 | 0.25 + 0.25 | 95 ± 3 | ++++ |
| Pc1 + Pc3 | 0.25 + 0.25 | 89 ± 5 | ++++ |
| Pc1 + Pc4 | 0.25 + 0.25 | 59 ± 6 | |
| Pc1 + Pc5 | 0.25 + 0.25 | 86 ± 7 | +++ |
| Pc1 + Pc6 | 0.25 + 0.25 | 53 ± 6 | |
| Pc2 + Pc3 | 0.25 + 0.25 | 94 ± 5 | ++++ |
| Pc2 + Pc4 | 0.25 + 0.25 | 81 ± 6 | +++ |
| Pc2 + Pc5 | 0.25 + 0.25 | 79 ± 4 | ++ |
| Pc2 + Pc6 | 0.25 + 0.25 | 48 ± 6 | |
| Pc2 + Pc7 | 0.25 + 0.25 | 85 ± 5 | +++ |
| Pc2 + Pc8 | 0.25 + 0.25 | 46 ± 5 | |
| Pc2 + Pc9 | 0.25 + 0.25 | 79 ± 5 | ++ |
| Pc2 + Pc10 | 0.25 + 0.25 | 82 ± 4 | +++ |
| Pc3 + Pc4 | 0.25 + 0.25 | 89 ± 6 | +++ |
| Pc3 + Pc5 | 0.25 + 0.25 | 80 ± 5 | +++ |

TABLE 6
Injection of two ladybird beetles with Pc1, Pc2,
Pc3, or Pc4 dsRNA (10 ng/mg fresh weight) did
not impact survivorship or reproductive rates.

| | | Mortality | |
|---|---|---|---|
| | | (dead/ | Reproductive rate² |
| Species injected | dsRNA injected | total)¹ | (Larvae/female) |
| Coccinella | gus | 2/24 | 14.4 ± 4.2 |
| septempunctata | | | |
| Coccinella | Pc1 | 1/23 | 13.7 ± 2.4 |
| septempunctata | | | |
| Coccinella | Pc2 | 2/24 | 15.2 ± 3.1 |
| septempunctata | | | |
| Coccinella | Pc3 | 0/21 | ND |
| septempunctata | | | |
| Coccinella | Pc4 | 2/24 | ND |
| septempunctata | | | |
| Coccinella | C. septempunctata | 19/21 | ND |
| septempunctata | vATPase | | |
| Hippodamia | gus | 2/19 | 8.3 ± 3.6 |
| tredecimpunctata | | | |
| Hippodamia | Pc1 | 1/18 | ND |
| tredecimpunctata | | | |
| Hippodamia | Pc2 | 2/21 | ND |
| tredecimpunctata | | | |
| Hippodamia | Pc3 | 1/17 | 7.8 ± 2.4 |
| tredecimpunctata | | | |
| Hippodamia | Pc4 | 2/18 | 9.1 ± 4.0 |
| tredecimpunctata | | | |

1. Between 25 and 30 insects were injected for each treatment, but those that died within 12 h of the injection were discarded, as the death was attributed to the injection injury, and not to dsRNA. No significant difference in mortality rates are seen between the negative control gus-dsRNA (a bacterial gene) and the insect dsRNAs (P>0.5, t test), except for the Coccinella septempunctata that were injected with their own species-specific dsRNA (P<0.05, t test).
2. Survivors of the dsRNA treatments were provided two mates and the number of larvae that hatched from eggs was counted and standardized to the number of females/container.

TABLE 7
Co-feeding dsRNase-specific dsRNA along with an insecticidal
dsRNA improves the efficacy of the insecticidal dsRNA. Flea
beetles (P. cruciferae and P. striolata) were fed either 1.0 ng/mm²
insecticidal dsRNA alone, or 0.5 ng/mm² of nuclease-dsRNA plus
0.5 ng/mm² of the insecticidal dsRNA. The percent mortality after
7 days of constant feeding on dsRNA-treated leaves are provided.
The values represent the means and standard errors for 3 replicates of 9
P. cruciferae beetles and 3 replicates of 6 P. striolata beetles.

| P. cruciferae | | | P. striolata | | |
|---|---|---|---|---|---|
| | % mortality | | | % mortality | |
| | | With | | | With |
| | No dsRNase | dsRNase | | No dsRNase | dsRNase |
| DsRNA | dsRNA | dsRNA | DsRNA | dsRNA | dsRNA |
| PC1 | 52 ± 4 | 86 ± 6 | PS1 | 48 ± 3 | 84 ± 6 |
| PC2 | 48 ± 5 | 88 ± 5 | PS2 | 52 ± 3 | 87 ± 7 |
| PC3 | 42 ± 6 | 83 ± 6 | PS3 | 48 ± 5 | 80 ± 3 |
| PC4 | 42 ± 4 | 86 ± 5 | PS4 | 42 ± 4 | 84 ± 5 |
| PC5 | 44 ± 6 | 90 ± 4 | PS5 | 40 ± 5 | 88 ± 6 |
| PC6 | 42 ± 5 | 79 ± 5 | PS6 | 42 ± 3 | 78 ± 5 |
| PC7 | 39 ± 5 | 74 ± 5 | PS7 | 37 ± 6 | 78 ± 3 |
| PC8 | 39 ± 3 | 69 ± 4 | PS8 | 37 ± 5 | 72 ± 4 |
| PC9 | 36 ± 3 | 64 ± 6 | PS9 | 34 ± 6 | 64 ± 5 |
| PC10 | 34 ± 4 | 59 ± 5 | PS10 | 30 ± 4 | 55 ± 3 |

### REFERENCES

1. Hannon G J (2002) RNA interference. Nature 418: 244-251
2. Reynolds A, et al. (2006). Induction of the interferon response by siRNA is cell type- and duplex length-dependent. RNA 12: 988-93.
3. Bolognesi R, et al. (2012) Characterizing the Mechanism of Action of Double-Stranded RNA Activity against Western Corn Rootworm (Diabrotica virgifera virgifera LeConte). PLoS ONE 7: e47534.
4. Hapairai L K, et al. (2017) Lure-and-Kill Yeast Interfering RNA Larvicides Targeting Neural Genes in the Human Disease Vector Mosquito Aedes aegypti. Scientific Reports 7: 13223.
5. Kim D H, Rossi J J (2008) RNAi mechanisms and applications. Biotechniques 44: 613-616
6. Baum J, et al. (2007) Control of coleopteran insect pests through RNA interference. Nature Biotech 25: 1322-1326.
7. Whyard S, Wong S, Singh A (2009) Ingested double-stranded RNAs can act as species-specific insecticides. Insect Biochem Mol Biol 39: 824-832.
8. Bachman P M, Bolognesi R, Moar W J, et al. (2013) Characterization of the spectrum of insecticidal activity of a double-stranded RNA with targeted activity against Western Corn Rootworm (Diabrotica virgifera virgifera LeConte). Transgenic Res. 22: 1207-1222.
9. Yang C, Preisser E L, Zhang H, Liu Y, Dai L, Pan H and Zhou X (2016) Selection of Reference Genes for RT-qPCR Analysis in Coccinella septempunctata to assess un-intended effects of RNAi transgenic plants. Front. Plant Sci. 7:1672.

1. A method of reducing feeding damage to a plant from a flea beetle of genus Phyllotreta comprising:
applying to a plant to be protected from flea beetles of the genus Phyllotreta an effective amount of a dsRNA to at least a portion of the plant to be protected, the nucleotide sequence of said dsRNA selected from the group consisting of:
(i) at least 21 consecutive nucleotides of the nucleotide sequence as set forth in SEQ ID No:1 (Pc1);
(ii) at least 21 consecutive nucleotides of the nucleotide sequence as set forth in SEQ ID No:2 (Pc2);
(iii) at least 21 consecutive nucleotides of the nucleotide sequence as set forth in SEQ ID No:3 (Pc3);
(iv) at least 21 consecutive nucleotides of the nucleotide sequence as set forth in SEQ ID No:4 (Pc4);
(v) at least 21 consecutive nucleotides of the nucleotide sequence as set forth in SEQ ID No:5 (Ps4);
(vi) at least 21 consecutive nucleotides of the nucleotide sequence as set forth in SEQ ID No:6 (Pc5);
(vii) at least 21 consecutive nucleotides of the nucleotide sequence as set forth in SEQ ID No:7 (Pc6);
(viii) at least 21 consecutive nucleotides of the nucleotide sequence as set forth in SEQ ID No:8 (Pc7);
(ix) at least 21 consecutive nucleotides of the nucleotide sequence as set forth in SEQ ID No:9 (Ps7);
(x) at least 21 consecutive nucleotides of the nucleotide sequence as set forth in SEQ ID No:10 (Pc8);
(xi) at least 21 consecutive nucleotides of the nucleotide sequence as set forth in SEQ ID No:11 (Pc9);
(xii) at least 21 consecutive nucleotides of the nucleotide sequence as set forth in SEQ ID No:12 (Pc10);
(xiii) at least 21 consecutive nucleotides of the nucleotide sequence as set forth in SEQ ID No:13 (Ps10);
and combinations thereof;
said dsRNA reducing feeding damage to the plant to be protected by reducing feeding activity of the flea beetles of the genus Phyllotreta following ingestion of said dsRNA by the flea beetles.

2. The method according to claim 1 wherein the effective amount of the dsRNA is co-administered with an effective amount of a nuclease inhibitor.

3. The method according to claim 2 wherein the nuclease inhibitor is a dsRNA targeted to a nuclease of flea beetles of species Phyllotreta.

4. The method according to claim 2 wherein the nuclease inhibitor is a nuclease inhibitor dsRNA selected from the group consisting of: at least 21 consecutive nucleotides of the nucleotide sequence as set forth in SEQ ID No: 14 (P. cruciferae DsRNase); and at least 21 consecutive nucleotides of the nucleotide sequence as set forth in SEQ ID No:15 (P. striolata DsRNase).

5. The method according to claim 1 wherein the dsRNA is applied to at least one leaf of the plant to be protected.

6. The method according to claim 1 wherein the dsRNA is applied to the plant to be protected at a concentration of at least about 0.1 ng per mm².

7. The method according to claim 1 wherein the flea beetle of the genus Phyllotreta is Phyllotreta cruciferae or Phyllotreta striolata.

8. The method according to claim 1 wherein the plant is a cruciferous plant.

9. The method according to claim 1 wherein the dsRNA is a mixture of a first dsRNA comprising at least 21 consecutive nucleotides of Pc1 and a second dsRNA comprising at least 21 consecutive nucleotides of Pc2 or Pc3.

10. The method according to claim 1 wherein the dsRNA is a mixture of a first dsRNA comprising at least 21 consecutive nucleotides of Pc3 and a second dsRNA comprising at least 21 consecutive nucleotides of Pc1, Pc2, Pc4 or Pc5.

11. The method according to claim 1 wherein the dsRNA is a mixture of a first dsRNA comprising at least 21 consecutive nucleotides of Pc2 and a second dsRNA comprising at least 21 consecutive nucleotides of Pc1, Pc3, Pc4, Pc5, Pc7, Pc9 or Pc10.

12. A method of protecting a plant from feeding damage by a flea beetle of genus Phyllotreta comprising
applying to a plant to be protected from flea beetles of the genus Phyllotreta an effective amount of a dsRNA to at least a portion of the plant to be protected, the nucleotide sequence of said dsRNA selected from the group consisting of:
(i) at least 21 consecutive nucleotides of the nucleotide sequence as set forth in SEQ ID No:1 (Pc1);
(ii) at least 21 consecutive nucleotides of the nucleotide sequence as set forth in SEQ ID No:2 (Pc2);
(iii) at least 21 consecutive nucleotides of the nucleotide sequence as set forth in SEQ ID No:3 (Pc3);
(iv) at least 21 consecutive nucleotides of the nucleotide sequence as set forth in SEQ ID No:4 (Pc4);
(v) at least 21 consecutive nucleotides of the nucleotide sequence as set forth in SEQ ID No:5 (Ps4);
(vi) at least 21 consecutive nucleotides of the nucleotide sequence as set forth in SEQ ID No:6 (Pc5);
(vii) at least 21 consecutive nucleotides of the nucleotide sequence as set forth in SEQ ID No:7 (Pc6);
(viii) at least 21 consecutive nucleotides of the nucleotide sequence as set forth in SEQ ID No:8 (Pc7);
(ix) at least 21 consecutive nucleotides of the nucleotide sequence as set forth in SEQ ID No:9 (Ps7);
(x) at least 21 consecutive nucleotides of the nucleotide sequence as set forth in SEQ ID No:10 (Pc8);
(xi) at least 21 consecutive nucleotides of the nucleotide sequence as set forth in SEQ ID No:11 (Pc9);
(xii) at least 21 consecutive nucleotides of the nucleotide sequence as set forth in SEQ ID No:12 (Pc10);
(xiii) at least 21 consecutive nucleotides of the nucleotide sequence as set forth in SEQ ID No:13 (Ps10);
and combinations thereof;
said dsRNA protecting the plant to be protected by reducing feeding activity of the flea beetles of the genus Phyllotreta following ingestion of said dsRNA by the flea beetles of the genus Phyllotreta.

13. The method according to claim 12 wherein the effective amount of the dsRNA is co-administered with an effective amount of a nuclease inhibitor.

14. The method according to claim 13 wherein the nuclease inhibitor is a dsRNA targeted to a nuclease of flea beetles of species Phyllotreta.

15. The method according to claim 13 wherein the nuclease inhibitor is a nuclease inhibitor dsRNA selected from the group consisting of: at least 21 consecutive nucleotides of the nucleotide sequence as set forth in SEQ ID No: 14 (P. cruciferae DsRNase); and at least 21 consecutive nucleotides of the nucleotide sequence as set forth in SEQ ID No:15 (P. striolata DsRNase).

16. The method according to claim 12 wherein the dsRNA is applied to at least one leaf of the plant to be protected.

17. The method according to claim 12 wherein the dsRNA is applied to the plant to be protected at a concentration of at least about 0.1 ng per mm².

18. The method according to claim 12 wherein the flea beetle of the genus Phyllotreta is Phyllotreta cruciferae or Phyllotreta striolata.

19. The method according to claim 12 wherein the plant is a cruciferous plant.

20. The method according to claim 12 wherein the dsRNA is a mixture of a first dsRNA comprising at least 21 consecutive nucleotides of Pc1 and a second dsRNA comprising at least 21 consecutive nucleotides of Pc2 or Pc3.

21. The method according to claim 12 wherein the dsRNA is a mixture of a first dsRNA comprising at least 21 consecutive nucleotides of Pc3 and a second dsRNA comprising at least 21 consecutive nucleotides of Pc1, Pc2, Pc4 or Pc5.

22. The method according to claim 12 wherein the dsRNA is a mixture of a first dsRNA comprising at least 21 consecutive nucleotides of Pc2 and a second dsRNA comprising at least 21 consecutive nucleotides of Pc1, Pc3, Pc4, Pc5, Pc7, Pc9 or Pc10.

23. A method of killing flea beetles of genus Phyllotreta comprising:
applying to a flea beetle of the genus Phyllotreta food source an effective amount of a dsRNA to at least a portion of the food source, the nucleotide sequence of said dsRNA selected from the group consisting of:
(i) at least 21 consecutive nucleotides of the nucleotide sequence as set forth in SEQ ID No:1 (Pc1);
(ii) at least 21 consecutive nucleotides of the nucleotide sequence as set forth in SEQ ID No:2 (Pc2);
(iii) at least 21 consecutive nucleotides of the nucleotide sequence as set forth in SEQ ID No:3 (Pc3);
(iv) at least 21 consecutive nucleotides of the nucleotide sequence as set forth in SEQ ID No:4 (Pc4);
(v) at least 21 consecutive nucleotides of the nucleotide sequence as set forth in SEQ ID No:5 (Ps4);
(vi) at least 21 consecutive nucleotides of the nucleotide sequence as set forth in SEQ ID No:6 (Pc5);
(vii) at least 21 consecutive nucleotides of the nucleotide sequence as set forth in SEQ ID No:7 (Pc6);
(viii) at least 21 consecutive nucleotides of the nucleotide sequence as set forth in SEQ ID No:8 (Pc7);
(ix) at least 21 consecutive nucleotides of the nucleotide sequence as set forth in SEQ ID No:9 (Ps7);
(x) at least 21 consecutive nucleotides of the nucleotide sequence as set forth in SEQ ID No:10 (Pc8);
(xi) at least 21 consecutive nucleotides of the nucleotide sequence as set forth in SEQ ID No:11 (Pc9);
(xii) at least 21 consecutive nucleotides of the nucleotide sequence as set forth in SEQ ID No:12 (Pc10);
(xiii) at least 21 consecutive nucleotides of the nucleotide sequence as set forth in SEQ ID No:13 (Ps10);
and combinations thereof;
said dsRNA killing the flea beetles of the genus Phyllotreta following ingestion of the food source comprising the dsRNA by the flea beetles.

24. The method according to claim 23 wherein the effective amount of the dsRNA is co-administered with an effective amount of a nuclease inhibitor.

25. The method according to claim 24 wherein the nuclease inhibitor is a dsRNA targeted to a nuclease of flea beetles of species Phyllotreta.

26. The method according to claim 24 wherein the nuclease inhibitor is a nuclease inhibitor dsRNA selected from the group consisting of: at least 21 consecutive nucleotides of the nucleotide sequence as set forth in SEQ ID No: 14 (P. cruciferae DsRNase); and at least 21 consecutive nucleotides of the nucleotide sequence as set forth in SEQ ID No:15 (P. striolata DsRNase).

27. The method according to claim 23 wherein the dsRNA is applied to the food source at a concentration of at least about 0.1 ng per mm².

28. The method according to claim 23 wherein the flea beetle of the genus Phyllotreta is Phyllotreta cruciferae or Phyllotreta striolata.

29. The method according to claim 23 wherein the plant is a cruciferous plant.

30. The method according to claim 23 wherein the dsRNA is a mixture of a first dsRNA comprising at least 21 consecutive nucleotides of Pc1 and a second dsRNA comprising at least 21 consecutive nucleotides of Pc2 or Pc3.

31. The method according to claim 23 wherein the dsRNA is a mixture of a first dsRNA comprising at least 21 consecutive nucleotides of Pc3 and a second dsRNA comprising at least 21 consecutive nucleotides of Pc1, Pc2, Pc4 or Pc5.

32. The method according to claim 23 wherein the dsRNA is a mixture of a first dsRNA comprising at least 21 consecutive nucleotides of Pc2 and a second dsRNA comprising at least 21 consecutive nucleotides of Pc1, Pc3, Pc4, Pc5, Pc7, Pc9 or Pc10.
